(19) United States
(12) Patent Application Publication
(10) Pub. No.: US 2002/0026088 A1
(43) Pub. Date: Feb. 28, 2002

(54) Acetaldehyde dehydration to produce ethyne

(76) Inventor: Christian Everett, Springfield, IL (US)

Correspondence Address: CHRISTIAN EVERETT, 2745 SOUTH 6TH ST. #106, SPRINGFIELD, IL, 62703, US

(21) Appl. No.: 09/910,248

(22) Filed: Jul. 20, 2001

Related U.S. Application Data
(60) Provisional application No. 60/228,828, filed on Aug. 28, 2000.

(51) Int. Cl. C07C 2/02; C07C 4/02; C07C 11/00

(52) U.S. Cl. 585/539

(57) ABSTRACT

A process of dehydrating acetaldehyde to produce ethyne comprising passing acetaldehyde in the gas phase over a dehydration catalyst such as aluminum oxide which may be promoted with mercuric sulfate at a temperature of approximately 600 degrees Centigrade to produce ethyne and water and a cooling zone following the reaction zone.

This method having the advantage of ease of separation of ethyne from the unreacted acetaldehyde and co-product water by simple condensation whereby the ethyne remains gaseous while the other compounds liquify and is thereby separated in essentially pure form.

### BACKGROUND-FIELD OF INVENTION

This invention relates to the production of ethyne (acetylene).

### BACKGROUND-DESCRIPTION OF PRIOR ART

Ethyne has been produced by the high temperature pyrolysis of ethene U.S. Pat. No. 5,942,652) and methane which is currently the industry standard. The disadvantage of this process is the high reaction temperature (1200 degrees Centigrade), low yields (30%) and the difficulty of separating the ethyne from the reactant methane and co-product hydrogen which results in the need for an elaborate process whereby the gasses are isolated by their different solubilities in various organic liquids. All this contributes to the price of ethyne being $6 per pound ($36 per gallon).

### OBJECTS AND ADVANTAGES

Several objects and advantages of the present invention are:

(A) Lower reaction temperature.

(B) Higher yield.

(C) Ease of separation of product from reactant and co-product.

(D) Low cost feedstock acetaldehyde (26 cents per pound 6/2000) which is itself produced from ethanol.

### SUMMARY

In accordance with the present invention a process to produce ethyne by the dehydration of acetaldehyde.

### DESCRIPTION

Acetaldehyde in the gas phase is passed through a reaction zone containing a dehydration catalyst such as aluminum oxide which may be promoted with mercuric sulfate at a temperature of approximately 600 degrees Centigrade to produce ethyne and water and a cooling zone to reduce the temperature of the gas stream to approximately 100 degrees Centigrade. The gas stream is then chilled to approximately 0 degrees Centigrade, causing the water and unreacted acetaldehyde to liquify whereby ethyne containing some acetaldehyde is obtained a gas. The ethyne may be further purified by reducing the temperature to −20 degrees Centigrade, whereby essentially all the acetaldehyde is removed.

Conclusion, Ramification and Scope

Accordingly, the reader will see the production of ethyne by the dehydration of acetaldehyde is a more convenient process than current methods. Furthermore the process has additional advantages in that

ultimate feedstock ethanol may be produces from renewable resources.

production plant need not be located at methane production site as acetaldehyde and ethanol may be shipped by truck, which methane cannot.

ultimate cost of ethyne will be less than by existing processes.

Although the description contains specifitys, these should not be construed as limiting the scope of the invention, but merely providing illustrations of the presently preferred embodiments of the invention. For example, higher or lower temperatures may be used or the catalyst may be any of a number of metal oxides promoted by any of a number of IIB metal salts.

Thus the scope of the invention should be determined by the appended claims and their legal equivalents, rather than by the examples given.

I claim:

1. A method for producing ethyne, comprising the steps of:
A: Passing acetaldehyde through a dehydration catalyst at elevated temperatures
B: Cooling resulting gas stream to liquify co-products and unreacted acetaldehyde whereby ethyne is obtained in essentially pure form.

2. A method for producing substituted ethynes, comprising the steps of:
A: Passing a mono-alpha substituted acetaldehyde through a dehydration catalyst at elevated temperatures.
B: Cooling resulting gas stream to liquify co-products and unreacted mono-alpha substituted acetaldehyde whereby substituted ethyne is obtained in essentially pure form.

3. A method for producing substituted ethynes comprising the steps of:
A: Passing a carbonyl-hydrogen substituted acetaldehyde through a dehydration catalyst at elevated temperatures.
B: Cooling resulting gas stream to liquify co-products and unreacted carbonyl-hydrogen substituted acetaldehyde whereby substituted ethyne is obtained in essentially pure form.
